SEQUENCE LISTING (2 sequences)

SEQ ID NO: 1 - DNA, 50 bp, Artificial Sequence
  16S_V3_F
  misc_feature (42): a, c, g, or t
tcgtcggcagcgtcagatgtgtataagagacagcctacgggnggcwgcag

SEQ ID NO: 2 - DNA, 55 bp, Artificial Sequence
  16S_V4_R
gtctcgtgggctcggagatgtgtataagagacaggactachvgggtatctaatcc

(19) United States
(12) Patent Application Publication
(10) Pub. No.: US 2023/0132988 A1
(43) Pub. Date: May 4, 2023

(54) METHOD FOR DIAGNOSING AUTISM BY ANALYZING BACTERIAL METAGENOME

(71) Applicant: MD HEALTHCARE INC., Seoul (KR)

(72) Inventor: Yoon-Keun KIM, Paju-si, Gyeonggi-do (KR)

(73) Assignee: MD HEALTHCARE INC., Seoul (KR)

(21) Appl. No.: 16/616,214

(22) PCT Filed: Apr. 25, 2018
(86) PCT No.: PCT/KR2018/004795

§ 371 Date: Nov. 22, 2019

(30) Foreign Application Priority Data
May 26, 2017 (KR) 10-2017-0065329
Apr. 24, 2018 (KR) 10-2018-0047608

(51) Int. Cl. C12Q 1/689; C12Q 1/6883

(52) U.S. Cl. CPC C12Q 1/689; C12Q 1/6883; C12Q 1/6844

(57) ABSTRACT

Provided are a method of diagnosing autism by performing a bacterial metagenomic analysis using normal individual-derived and subject-derived samples to analyze an increase or decrease in the content of specific bacteria-derived extracellular vesicles. The method predicts the risk of autism onset in advance through the metagenomic analysis of bacteria-derived extracellular vesicles using human body-derived samples, and it is possible to delay the onset time or prevent the onset of autism through proper management following early diagnosis and prediction of an autism risk group, and even after the onset, it is possible to diagnose autism early, thereby lowering the incidence of autism and increasing therapeutic effects.

### CROSS-REFERENCE TO RELATED APPLICATIONS

This application is a 371 of PCT/KR2018/004795, filed Apr. 25, 2018, which claims the benefit of priority from Korean Patent Application No. 10-2017-0065329, filed May 26, 2017 and Korean Patent Application No. 10-2018-0047608, filed Apr. 24, 2018, the contents of each of which are incorporated herein by reference in its entirety.

### SEQUENCE LISTING

The Sequence Listing submitted in text format (.txt) filed on Apr. 13, 2020, named “SequenceListing.txt”, created on Mar. 25, 2020 (729 bytes), is incorporated herein by reference.

### TECHNICAL FIELD

The present invention relates to a method for diagnosing autism through a bacterial metagenomic analysis and, more specifically, to a method of diagnosing autism, and the like by performing a bacterial metagenomic analysis using normal individual-derived and subject-derived samples to analyze an increase or decrease in the content of specific bacteria-derived extracellular vesicles.

### BACKGROUND ART

Autism refers to a neurodevelopmental disorder that reduces the ability to understand communication and social interactions. The cause of this symptom has not been clearly elucidated yet, but several researchers speculate that the cause is not an emotional cause, but a genetic developmental disorder. The probability of onset is one in every 1,000 people, and the ratio of men to women is approximately 4:1 according to US standards. A 2011 survey shows that the prevalence rate of autism in Korea is 2% or more, and thus has received attention. Autism is generally referred to as ‘autism spectrum disorder’, and this disorder has been discussed in three separate sections in the academic world. The first is autism (Kanner's Syndrome) referring to children as defined by Leo Kanner, the second is Asperger's Syndrome discussed by Hans Asperger, and the last is high functional autism including Savant Syndrome.

Growing babies generally show social activities such as staring at people, paying attention to voices, grasping fingers, or laughing or smiling. However, autistic babies do not like face to face interaction, and suffer from great difficulties in understanding human societal interactions. In relation to communication, ordinary babies say words on their first birthday, react when their names are called, show actions by indicating toys with their fingers when the babies want the toys, whereas autistic babies choose a different path in the development of communication. Even though some people with autism have a sufficient ability to read and write, people with autism remain silent throughout their lives, and instead, they prefer other methods, that is, methods such as painting or sign language.

In relation to the cause of autism, some theories asserted that autism seemed to be acquired at an early stage of life, but currently, it is predominantly thought that autism is caused by a congenital cause. Usually, abnormalities in brain structure, genetic defects, and abnormalities in neurotransmitters have been identified as the cause of autism. Further, fetal alcohol syndrome, in which a kid is adversely affected by a pregnant woman who likes drinking, was also identified as a cause of autism. However, there is still an ongoing debate about the exact causal factors.

A microbiota or microbiome is a microbial community that includes bacteria, archaea, and eukaryotes present in a given habitat. The intestinal microbiota is known to play a vital role in human's physiological phenomena and significantly affect human health and diseases through interactions with human cells. Bacteria coexisting in human bodies secrete nanometer-sized vesicles to exchange information about genes, proteins, and the like with other cells. The mucous membranes form a physical barrier membrane that does not allow particles with the size of 200 nm or more to pass therethrough, and thus bacteria symbiotically living in the mucous membranes are unable to pass therethrough, but bacteria-derived extracellular vesicles have a size of approximately 100 nm or less and thus relatively freely pass through the mucous membranes and are absorbed into the human body.

Metagenomics, also called environmental genomics, may be analytics for metagenomic data obtained from samples collected from the environment (Korean Patent Publication No. 2011-073049). Recently, the bacterial composition of human microbiota has been listed using a method based on 16s ribosomal RNA (16s rRNA) base sequences, and 16s rDNA base sequences, which are genes of 16s ribosomal RNA, are analyzed using a next generation sequencing (NGS) platform. However, in the onset of autism, identification of causative factors of autism through metagenomic analysis of bacteria-derived vesicles isolated from a human-derived substance, such as urine and the like, and a method of predicting autism have never been reported.

### DISCLOSURE

### Technical Problem

The present inventors extracted genes from bacteria-derived extracellular vesicles present in urine as normal individual-derived and subject-derived samples and performed a metagenomic analysis in this regard in order to diagnose the causal factors and risk of autism in advance, and as a result, identified bacteria-derived extracellular vesicles which may act as a causal factor of autism, thereby completing the present invention based on this.

Therefore, an object of the present invention is to provide a method of providing information for diagnosing autism, a method of diagnosing autism, a method of predicting the risk of autism onset, and the like through the metagenomic analysis of bacteria-derived extracellular vesicles.

However, the technical goals of the present invention are not limited to the aforementioned goals, and other unmentioned technical goals will be clearly understood by those of ordinary skill in the art from the following description.

### Technical Solution

To achieve the above-described object of the present invention, there is provided a method of providing information for autism diagnosis, comprising the following processes:

(a) extracting DNAs from extracellular vesicles isolated from normal individual and subject samples;

(b) performing polymerase chain reaction (PCR) on the extracted DNA using a pair of primers having SEQ ID NO: 1 and SEQ ID NO: 2; and

(c) comparing an increase or decrease in content of bacteria-derived extracellular vesicles of the subject-derived sample with that of a normal individual-derived sample through sequencing of a product of the PCR.

The present invention also provides a method of diagnosing autism, comprising the following processes:

(a) extracting DNAs from extracellular vesicles isolated from normal individual and subject samples;

(b) performing polymerase chain reaction (PCR) on the extracted DNA using a pair of primers having SEQ ID NO: 1 and SEQ ID NO: 2; and

(c) comparing an increase or decrease in content of bacteria-derived extracellular vesicles of the subject-derived sample with that of a normal individual-derived sample through sequencing of a product of the PCR.

The present invention also provides a method of predicting a risk for autism, comprising the following processes:

(a) extracting DNAs from extracellular vesicles isolated from normal individual and subject samples;

(b) performing polymerase chain reaction (PCR) on the extracted DNA using a pair of primers having SEQ ID NO: 1 and SEQ ID NO: 2; and

(c) comparing an increase or decrease in content of bacteria-derived extracellular vesicles of the subject-derived sample with that of a normal individual-derived sample through sequencing of a product of the PCR.

In one embodiment of the present invention, the normal individual and subject samples may be urine.

In another embodiment of the present invention, process (c) may comprise comparing an increase or decrease in content of extracellular vesicles derived from one or more bacteria selected from the group consisting of the phylum Armatimonadetes and the phylum Thermi.

In another embodiment of the present invention, process (c) may comprise comparing an increase or decrease in content of extracellular vesicles derived from one or more bacteria selected from the group consisting of the class Fimbriimonadia, the class Alphaproteobacteria, and the class Deinococci.

In another embodiment of the present invention, process (c) may comprise comparing an increase or decrease in content of extracellular vesicles derived from one or more bacteria selected from the group consisting of the order Fimbriimonadales, the order Rhizobiales, and the order Sphingomonadales, and the order Desulfovibrionales.

In another embodiment of the present invention, process (c) may comprise comparing an increase or decrease in content of extracellular vesicles derived from one or more bacteria selected from the group consisting of the family Fimbriimonadaceae, the family Rhizobiaceae, the family Alcaligenaceae, the family Sphingomonadaceae, and the family Desulfovibrionaceae.

In another embodiment of the present invention, process (c) may comprise comparing an increase or decrease in content of extracellular vesicles derived from one or more bacteria selected from the group consisting of the genus Fimbriimonas, the genus Achromobacter, the genus Roseateles, the genus Agrobacterium, the genus Sphingomonas, the genus Kocuria, the genus Desulfovibrio, the genus Halomonas, and the genus Jeotgalicoccus.

In another embodiment of the present invention, process (c) may comprise comparing an increase or decrease in content of extracellular vesicles derived from one or more bacteria selected from the group consisting of the phylum Armatimonadetes and the phylum Thermi;

extracellular vesicles derived from one or more bacteria selected from the group consisting of the class Fimbriimonadia, the class Alphaproteobacteria, and the class Deinococci;

extracellular vesicles derived from one or more bacteria selected from the group consisting of the order Fimbriimonadales, the order Rhizobiales, and the order Sphingomonadales, and the order Desulfovibrionales;

extracellular vesicles derived from one or more bacteria selected from the group consisting of the family Fimbriimonadaceae, the family Rhizobiaceae, the family Alcaligenaceae, the family Sphingomonadaceae, and the family Desulfovibrionaceae; or

extracellular vesicles derived from one or more bacteria selected from the group consisting of the genus Fimbriimonas, the genus Achromobacter, the genus Roseateles, the genus Agrobacterium, the genus Sphingomonas, the genus Kocuria, the genus Desulfovibrio, the genus Halomonas, and the genus Jeotgalicoccus.

As still another embodiment of the present invention, in process (c), in comparison with the normal individual-derived sample, it is possible to diagnose an increase in the content of the following as autism:

extracellular vesicles derived from bacteria of the phylum Thermi,

extracellular vesicles derived from bacteria of the class Deinococci,

extracellular vesicles derived from bacteria of the order Desulfovibrionales,

extracellular vesicles derived from bacteria of the family Desulfovibrionaceae, or

extracellular vesicles derived from one or bacteria selected from the group consisting of the genus Kocuria, the genus Desulfovibrio, the genus Halomonas, and the genus Jeotgalicoccus.

As still another embodiment of the present invention, in process (c), in comparison with the normal individual-derived sample, it is possible to diagnose a decrease in the content of the following as autism:

extracellular vesicles derived from bacteria of the phylum Armatimonadetes,

extracellular vesicles derived from one or more bacteria selected from the group consisting of the class Fimbriimonadia and the class Alphaproteobacteria,

extracellular vesicles derived from one or more bacteria selected from the group consisting of the order Fimbriimonadales, the order Rhizobiales, and the order Sphingomonadales,

extracellular vesicles derived from one or more bacteria selected from the group consisting of the family Fimbriimonadaceae, the family Rhizobiaceae, the family Alcaligenaceae, and the family Sphingomonadaceae, or

extracellular vesicles derived from one or more bacteria selected from the group consisting of the genus Fimbriimonas, the genus Achromobacter, the genus Roseateles, the genus Agrobacterium, and the genus Sphingomonas.

### Advantageous Effects

The extracellular vesicles secreted from microorganisms such as bacteria and archaebacteria present in the environment are absorbed into the human body and thus may directly affect the occurrence of inflammation, and before symptoms appear, it is difficult to early diagnose autism characterized by inflammation, and thus it is difficult to efficiently treat autism. Therefore, by diagnosing the risk of autism onset in advance through the metagenomic analysis of bacteria-derived extracellular vesicles using human body-derived samples according to the present invention, it is possible to diagnose and predict an autism risk group early, and to delay the onset time or prevent the onset of autism through proper management. In addition, even after the onset, since it is possible to diagnose autism early, there are advantages in that it is possible to lower the incidence of autism and increase therapeutic effects, and it is possible to ameliorate the progression of the disease or prevent recurrence by avoiding exposure to causal factors through a metagenomic analysis in a patient diagnosed with autism.

### DESCRIPTION OF DRAWINGS

FIG. 1A illustrates images showing the distribution pattern of bacteria and extracellular vesicles over time after intestinal bacteria and bacteria-derived extracellular vesicles (EVs) were orally administered to mice, and FIG. 1B illustrates images showing the distribution pattern of bacteria and EVs after being orally administered to mice and, at 12 hours, blood and various organs were extracted.

FIG. 2 is a result showing the distribution of bacteria-derived extracellular vesicles (EVs), which is significant in diagnostic performance at the phylum level by isolating bacteria-derived vesicles from urine of a patient with autism and a normal individual, and then performing a metagenomic analysis.

FIG. 3 is a result showing the distribution of bacteria-derived extracellular vesicles (EVs), which is significant in diagnostic performance at the class level by isolating bacteria-derived vesicles from urine of a patient with autism and a normal individual, and then performing a metagenomic analysis.

FIG. 4 is a result showing the distribution of bacteria-derived extracellular vesicles (EVs), which is significant in diagnostic performance at the order level by isolating bacteria-derived vesicles from urine of a patient with autism and a normal individual, and then performing a metagenomic analysis.

FIG. 5 is a result showing the distribution of bacteria-derived extracellular vesicles (EVs), which is significant in diagnostic performance at the family level by isolating bacteria-derived vesicles from urine of a patient with autism and a normal individual, and then performing a metagenomic analysis.

FIG. 6 is a result showing the distribution of bacteria-derived extracellular vesicles (EVs), which is significant in diagnostic performance at the genus level by isolating bacteria-derived vesicles from urine of a patient with autism and a normal individual, and then performing a metagenomic analysis.

### BEST MODE

The present invention relates to a method of diagnosing autism through bacterial metagenomic analysis. The inventors of the present invention extracted genes from bacteria-derived extracellular vesicles using a normal individual and a subject-derived sample, performed metagenomic analysis thereon, and identified bacteria-derived extracellular vesicles capable of acting as a causative factor of autism.

Therefore, the present invention provides a method of providing information for diagnosing autism, the method comprising:

(a) extracting DNAs from extracellular vesicles isolated from normal individual and subject samples;

(b) performing polymerase chain reaction (PCR) on the extracted DNA using a pair of primers having SEQ ID NO: 1 and SEQ ID NO: 2; and

(c) comparing an increase or decrease in content of bacteria-derived extracellular vesicles of the subject-derived sample with that of a normal individual-derived sample through sequencing of a product of the PCR.

The term “autism diagnosis” as used herein refers to determining whether a patient has a risk for autism, whether the risk for autism is relatively high, or whether autism has already occurred. The method of the present invention may be used to delay the onset of autism through special and appropriate care for a specific patient, which is a patient having a high risk for autism or prevent the onset of autism. In addition, the method may be clinically used to determine treatment by selecting the most appropriate treatment method through early diagnosis of autism.

The term “metagenome” as used herein refers to the total of genomes including all viruses, bacteria, fungi, and the like in isolated regions such as soil, the intestines of animals, and the like, and is mainly used as a concept of genomes that explains identification of many microorganisms at once using a sequencer to analyze non-cultured microorganisms. In particular, a metagenome does not refer to a genome of one species, but refers to a mixture of genomes, including genomes of all species of an environmental unit. This term originates from the view that, when defining one species in a process in which biology is advanced into omics, various species as well as existing one species functionally interact with each other to form a complete species. Technically, it is the subject of techniques that analyzes all DNAs and RNAs regardless of species using rapid sequencing to identify all species in one environment and verify interactions and metabolism. In the present invention, bacterial metagenomic analysis is performed using bacteria-derived extracellular vesicles isolated from, for example, serum.

In the present invention, the normal individual and subject sample may be urine, but the present invention is not limited thereto.

In an embodiment of the present invention, metagenomic analysis is performed on the bacteria-derived extracellular vesicles, and bacteria-derived extracellular vesicles capable of acting as a cause of the onset of Parkinson's disease were actually identified by analysis at phylum, class, order, family, and genus levels.

More particularly, in one embodiment of the present invention, as a result of performing bacterial metagenomic analysis on extracellular vesicles present in subject-derived urine samples at a phylum level, the content of extracellular vesicles derived from bacteria belonging to the phylum Armatimonadetes and the phylum Thermi was significantly different between autism patients and normal individuals (see Example 4).

More particularly, in one embodiment of the present invention, as a result of performing bacterial metagenomic analysis on extracellular vesicles present in subject-derived urine samples at a class level, the content of extracellular vesicles derived from bacteria belonging to the class Fimbriimonadia, the class Alphaproteobacteria, and the class Deinococci was significantly different between autism patients and normal individuals (see Example 4).

More particularly, in one embodiment of the present invention, as a result of performing bacterial metagenomic analysis on extracellular vesicles present in subject-derived urine samples at an order level, the content of extracellular vesicles derived from bacteria belonging to the order Fimbriimonadales, the order Rhizobiales, the order Sphingomonadales, and the order Desulfovibrionales was significantly different between autism patients and normal individuals (see Example 4).

More particularly, in one embodiment of the present invention, as a result of performing bacterial metagenomic analysis on extracellular vesicles present in subject-derived urine samples at a family level, the content of extracellular vesicles derived from bacteria belonging to the family Fimbriimonadaceae, the family Rhizobiaceae, the family Alcaligenaceae, the family Sphingomonadaceae, and the family Desulfovibrionaceae was significantly different between autism patients and normal individuals (see Example 4).

More particularly, in one embodiment of the present invention, as a result of performing bacterial metagenomic analysis on extracellular vesicles present in subject-derived urine samples at a genus level, the content of extracellular vesicles derived from bacteria belonging to the genus Fimbriimonas, the genus Achromobacter, the genus Roseateles, the genus Agrobacterium, the genus Sphingomonas, the genus Kocuria, the genus Desulfovibrio, the genus Halomonas, and the genus Jeotgalicoccus was significantly different between autism patients and normal individuals (see Example 4).

Through the results of the examples, it was confirmed that distribution variables of the identified bacteria-derived extracellular vesicles could be usefully used for the prediction of the onset of autism.

### [Mode of the Invention]

Hereinafter, the present invention will be described with reference to exemplary examples to aid in understanding of the present invention. However, these examples are provided only for illustrative purposes and are not intended to limit the scope of the present invention.

### EXAMPLES

### Example 1. Analysis of In Vivo Absorption, Distribution, and Excretion Patterns of Intestinal Bacteria and Bacteria-Derived Extracellular Vesicles

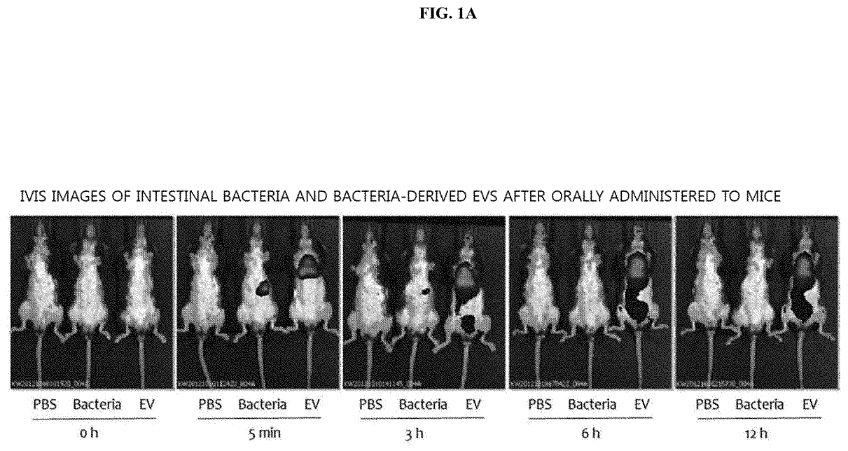

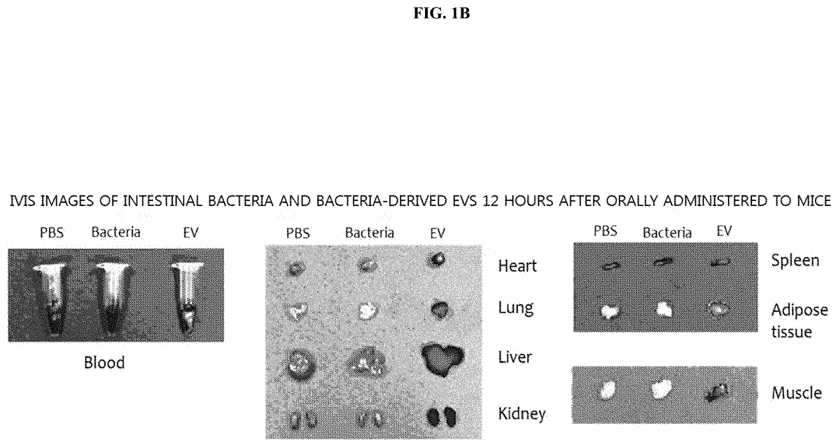

To evaluate whether intestinal bacteria and bacteria-derived extracellular vesicles are systematically absorbed through the gastrointestinal tract, an experiment was conducted using the following method. More particularly, 50 μg of each of intestinal bacteria and the bacteria-derived extracellular vesicles (EVs), labeled with fluorescence, were orally administered to the gastrointestinal tracts of mice, and fluorescence was measured at 0 h, and after 5 min, 3 h, 6 h, and 12 h. As a result of observing the entire images of mice, as illustrated in FIG. 1A, the bacteria were not systematically absorbed when administered, while the bacteria-derived EVs were systematically absorbed at 5 min after administration, and, at 3 h after administration, fluorescence was strongly observed in the bladder, from which it was confirmed that the EVs were excreted via the urinary system, and were present in the bodies up to 12 h after administration.

After intestinal bacteria and intestinal bacteria-derived extracellular vesicles were systematically absorbed, to evaluate a pattern of invasion of intestinal bacteria and the bacteria-derived EVs into various organs in the human body after being systematically absorbed, 50 μg of each of the bacteria and bacteria-derived EVs, labeled with fluorescence, were administered using the same method as that used above, and then, at 12 h after administration, blood, the heart, the lungs, the liver, the kidneys, the spleen, adipose tissue, and muscle were extracted from each mouse. As a result of observing fluorescence in the extracted tissues, as illustrated in FIG. 1B, it was confirmed that the intestinal bacteria were not absorbed into each organ, while the bacteria-derived EVs were distributed in the blood, heart, lungs, liver, kidneys, spleen, adipose tissue, and muscle.

### Example 2. Vesicle Isolation and DNA Extraction from Urine

To isolate extracellular vesicles and extract DNA, from urine, first, urine was added to a 10 ml tube and centrifuged at 3,500×g and 4° C. for 10 min to precipitate a suspension, and only a supernatant was collected, which was then placed in a new 10 ml tube. The collected supernatant was filtered using a 0.22 μm filter to remove bacteria and impurities, and then placed in centripreigugal filters (50 kD) and centrifuged at 1500×g and 4° C. for 15 min to discard materials with a smaller size than 50 kD, and then concentrated to 10 ml. Once again, bacteria and impurities were removed therefrom using a 0.22 μm filter, and then the resulting concentrate was subjected to ultra-high speed centrifugation at 150,000×g and 4° C. for 3 hours by using a Type 90ti rotor to remove a supernatant, and the agglomerated pellet was dissolved with phosphate-buffered saline (PBS), thereby obtaining vesicles.

100 μl of the extracellular vesicles isolated from the urine according to the above-described method was boiled at 100° C. to allow the internal DNA to come out of the lipid and then cooled on ice. Next, the resulting vesicles were centrifuged at 10,000×g and 4° C. for 30 minutes to remove the remaining suspension, only the supernatant was collected, and then the amount of DNA extracted was quantified using a NanoDrop sprectrophotometer. In addition, to verify whether bacteria-derived DNA was present in the extracted DNA, PCR was performed using 16s rDNA primers shown in Table 1 below.

TABLE 1

| Primer | | Sequence | SEQ ID NO. |
|---|---|---|---|
| 16S rDNA | 16S_V3_F | 5′-TCGTCGGCAGCGTC | 1 |
| | | AGATGTGTATAAGAG | |
| | | ACAGCCTACGGGNGG | |
| | | CWGCAG-3′ | |
| | 16S_V4_R | 5′-GTCTCGTGGGCTCG | 2 |
| | | GAGATGTGTATAAGA | |
| | | GACAGGACTACHVGG | |
| | | GTATCTAATCC-3′ | |

### Example 3. Metagenomic Analysis Using DNA Extracted from Urine

DNA was extracted using the same method as that used in Example 2, and then PCR was performed thereon using 16S rDNA primers shown in Table 1 to amplify DNA, followed by sequencing (Illumina MiSeq sequencer). The results were output as standard flowgram format (SFF) files, and the SFF files were converted into sequence files (.fasta) and nucleotide quality score files using GS FLX software (v2.9), and then credit rating for reads was identified, and portions with a window (20 bps) average base call accuracy of less than 99% (Phred score<20) were removed. After removing the low-quality portions, only reads having a length of 300 bps or more were used (Sickle version 1.33), and, for operational taxonomy unit (OTU) analysis, clustering was performed using UCLUST and USEARCH according to sequence similarity. In particular, clustering was performed based on sequence similarity values of 94% for genus, 90% for family, 85% for order, 80% for class, and 75% for phylum, and phylum, class, order, family, and genus levels of each OTU were classified, and bacteria with a sequence similarity of 97% or more were analyzed (QIIME) using 16S DNA sequence databases (108,453 sequences) of BLASTN and GreenGenes.

### Example 4. Autism Diagnostic Model Based on Metagenomic Analysis of Bacteria-Derived EVs Isolated from Urine

EVs were isolated from urine samples of 20 autism patients and 28 normal individuals, the two groups matched in age and gender, and then metagenomic sequencing was performed thereon using the method of Example 3. For the development of a diagnostic model, first, a strain exhibiting a p value of less than 0.05 between two groups in a t-test and a difference of two-fold or more between two groups was selected, and then an area under curve (AUC), sensitivity, and specificity, which are diagnostic performance indexes, were calculated by logistic regression analysis.

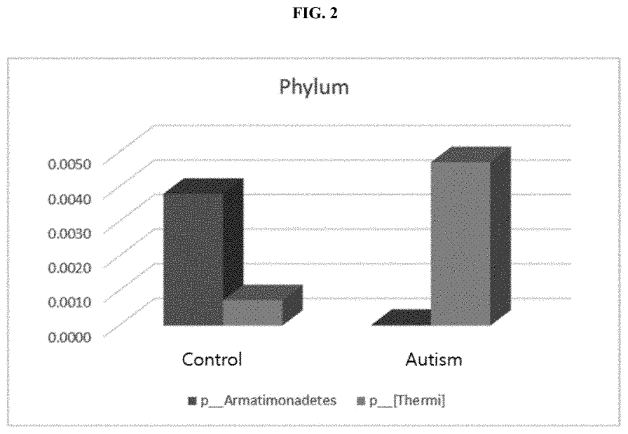

As a result of analyzing bacteria-derived EVs in urine at a phylum level, a diagnostic model developed using bacteria belonging to the phylum Armatimonadetes and the phylum Thermi as a biomarker exhibited significant diagnostic performance for autism (see Table 2 and FIG. 2).

TABLE 2

| | | | | | t-test | | | | | |
|---|---|---|---|---|---|---|---|---|---|---|
| | Control | | Autism | | p- | | | Accur- | sensi- | speci- |
| Taxon | Mean | SD | Mean | SD | value | Ratio | AUC | acy | tivity | ficity |
| p_ | 0.0038 | 0.0065 | 0.0000 | 0.0001 | 0.0046 | 0.00 | 0.74 | 0.73 | 0.57 | 0.95 |
| Armatimonadetes | | | | | | | | | | |
| p_Thermi | 0.0007 | 0.0015 | 0.0047 | 0.0052 | 0.0031 | 6.47 | 0.81 | 0.71 | 0.82 | 0.55 |

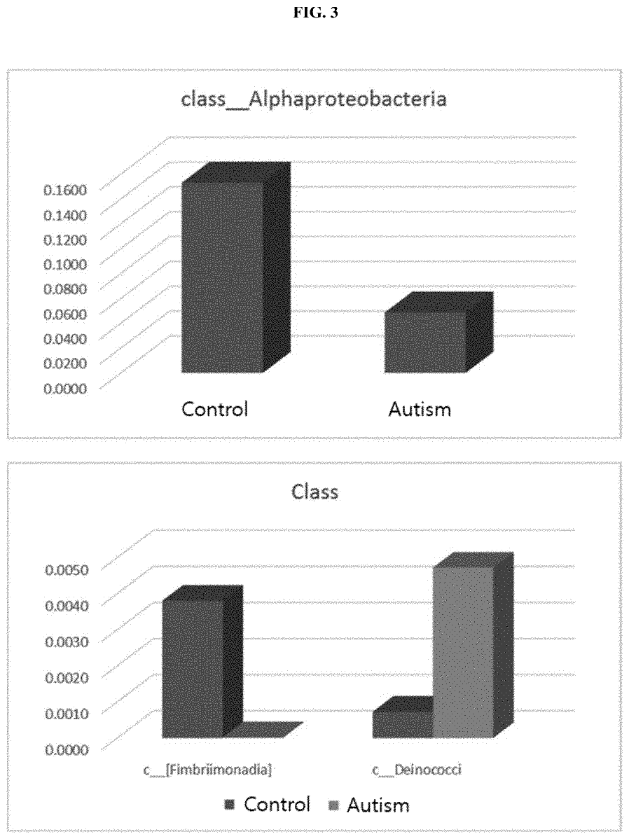

As a result of analyzing bacteria-derived EVs in urine at a class level, a diagnostic model developed using bacteria belonging to the class Fimbriimonadia, the class Alphaproteobacteria, and the class Deinococci as a biomarker exhibited significant diagnostic performance for autism (see Table 3 and FIG. 3).

TABLE 3

| | | | | | t-test | | | | | |
|---|---|---|---|---|---|---|---|---|---|---|
| | Control | | Autism | | p- | | | Accur- | sensi- | speci- |
| Taxon | Mean | SD | Mean | SD | value | Ratio | AUC | acy | tivity | ficity |
| c_ | 0.0038 | 0.0065 | 0.0000 | 0.0001 | 0.0046 | 0.00 | 0.74 | 0.73 | 0.57 | 0.95 |
| Fimbriimonadia | | | | | | | | | | |
| c_ | 0.1529 | 0.1278 | 0.0484 | 0.0258 | 0.0003 | 0.32 | 0.85 | 0.77 | 0.79 | 0.75 |
| Alphaproteobacteria | | | | | | | | | | |
| c_ | 0.0007 | 0.0015 | 0.0047 | 0.0052 | 0.0031 | 6.47 | 0.81 | 0.71 | 0.82 | 0.55 |
| Deinococci | | | | | | | | | | |

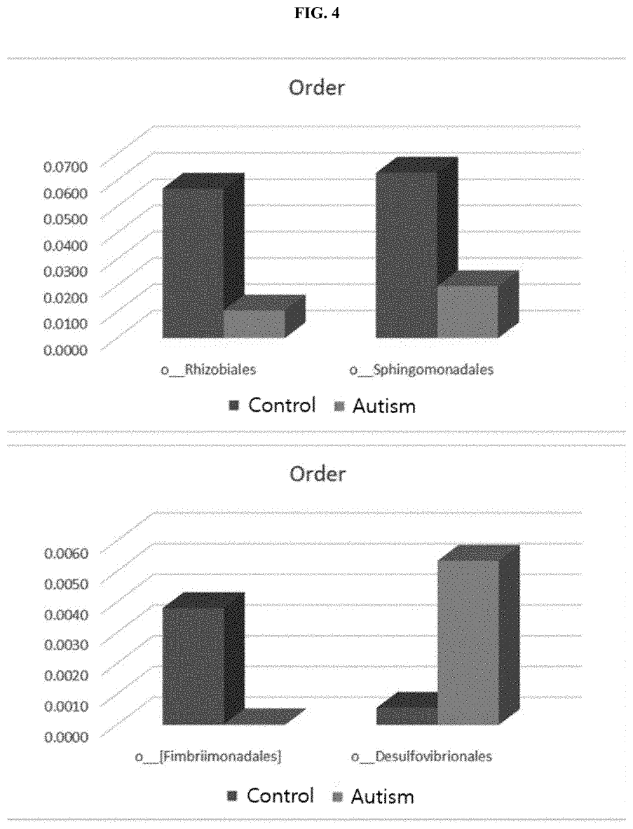

As a result of analyzing bacteria-derived EVs in urine at an order level, a diagnostic model developed using bacteria belonging to the order Fimbrilmonadales, the order Rhizobiales, the order Sphingomonadales, and the order Desulfovibrionales as a biomarker exhibited significant diagnostic performance for autism (see Table 4 and FIG. 4).

TABLE 4

| | | | | | t-test | | | | | |
|---|---|---|---|---|---|---|---|---|---|---|
| | Control | | Autism | | p- | | | Accur- | sensi- | speci- |
| Taxon | Mean | SD | Mean | SD | value | Ratio | AUC | acy | tivity | ficity |
| o_ | 0.0038 | 0.0065 | 0.0000 | 0.0001 | 0.0046 | 0.00 | 0.74 | 0.73 | 0.57 | 0.95 |
| Fimbriimonadales | | | | | | | | | | |
| o_Rhizobiales | 0.0569 | 0.0539 | 0.0105 | 0.0092 | 0.9001 | 0.19 | 091 | 0.83 | 0.86 | 0.80 |
| o_ | 0.0629 | 0.0642 | 0.0198 | 0.0159 | 0.0018 | 0.31 | 0.81 | 0.73 | 0.82 | 0.60 |
| Sphingomonadales | | | | | | | | | | |
| o_ | 0.0006 | 0.0014 | 0.0053 | 0.0059 | 0.0019 | 9.66 | 0.82 | 0.81 | 0.93 | 0.65 |
| Desulfovibnionales | | | | | | | | | | |

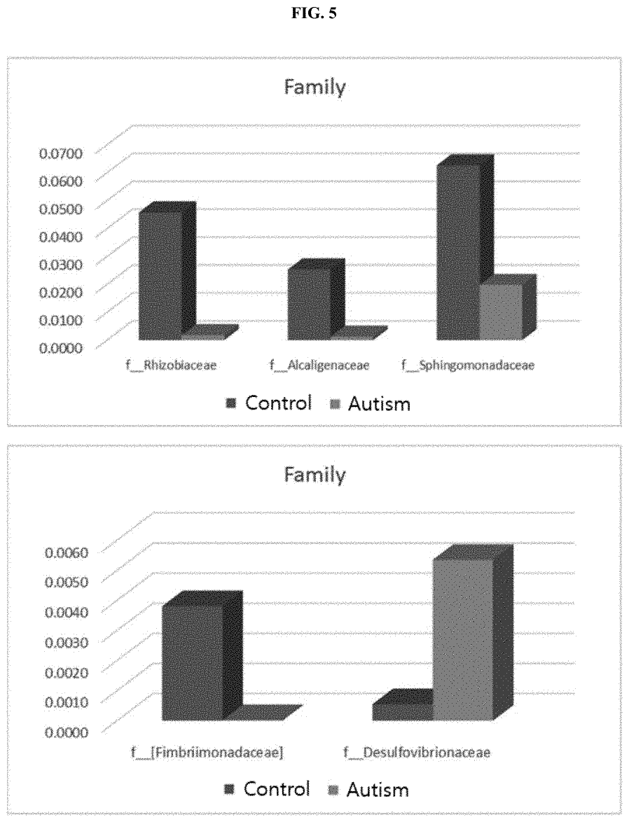

As a result of analyzing bacteria-derived EVs in urine at a family level, a diagnostic model developed using bacteria belonging to the family Fimbriimonadaceae, the family Rhizobiaceae, the family Alcaligenaceae, the family Sphingomonadaceae, and the family Desulfovibrionaceae as a biomarker exhibited significant diagnostic performance for autism (see Table 5 and FIG. 5).

TABLE 5

| | | | | | t-test | | | | | |
|---|---|---|---|---|---|---|---|---|---|---|
| | Control | | Autism | | p- | | | Accur- | sensi- | speci- |
| Taxon | Mean | SD | Mean | SD | value | Ratio | AUC | acy | tivity | ficity |
| f_ | 0.0038 | 0.0065 | 0.0000 | 0.0001 | 0.0046 | 0.00 | 0.74 | 0.73 | 0.57 | 0.95 |
| Fimbriimonadaceae | | | | | | | | | | |
| f_ | 0.0458 | 0.0504 | 0.0018 | 0.0028 | 0.0001 | 0.04 | 0.96 | 0.88 | 0.89 | 0.95 |
| Rhizobiaceae | | | | | | | | | | |
| f_ | 0.0253 | 0.0409 | 0.0012 | 0.0029 | 0.0044 | 0.05 | 0.79 | 0.75 | 0.82 | 0.65 |
| Alcaligenaceae | | | | | | | | | | |
| f_ | 0.0627 | 0.0642 | 0.0198 | 0.0159 | 0.0019 | 0.32 | 0.81 | 0.73 | 0.82 | 0.60 |
| Sphingomonadaceae | | | | | | | | | | |
| f_ | 0.0006 | 0.0014 | 0.0053 | 0.0059 | 0.0019 | 9.66 | 0.82 | 0.81 | 0.93 | 0.66 |
| Desulfovibrionaceae | | | | | | | | | | |

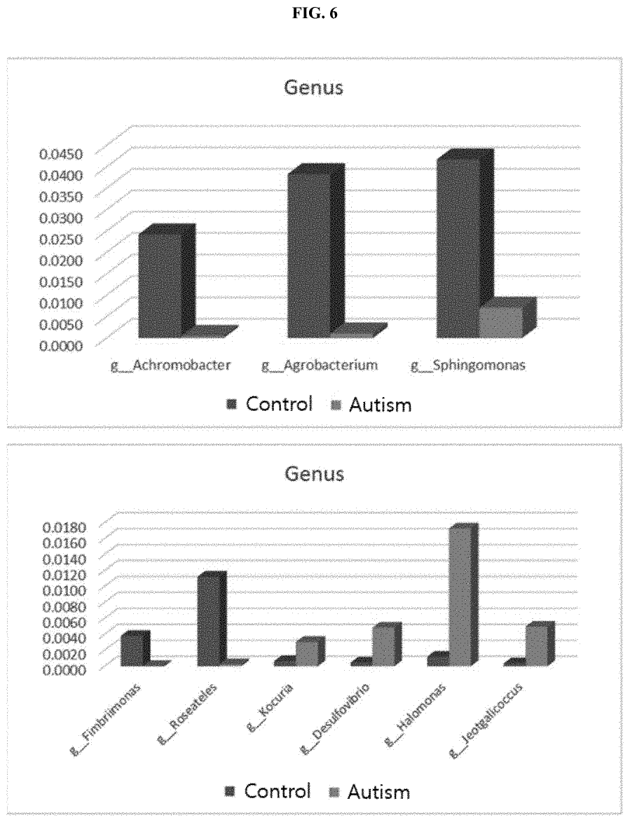

As a result of analyzing bacteria-derived EVs in urine at a genus level, a diagnostic model developed using bacteria belonging to the genus Fimbriimonas, the genus Achromobacter, the genus Roseateles, the genus Agrobacterium, the genus Sphingomonas, the genus Kocuria, the genus Desulfovibrio, the genus Halomonas, and the genus Jeotgalicoccus as a biomarker exhibited significant diagnostic performance for autism (see Table 6 and FIG. 6).

TABLE 6

| | | | | | t-test | | | | | |
|---|---|---|---|---|---|---|---|---|---|---|
| | Control | | Autism | | p- | | | Accur- | sensi- | speci- |
| Taxon | Mean | SD | Mean | SD | value | Ratio | AUC | acy | tivity | ficity |
| g_ | 0.0038 | 0.0065 | 0.0000 | 0.0001 | 0.0046 | 0.00 | 0.74 | 0,73 | 0.57 | 0.95 |
| Fimbriimonas | | | | | | | | | | |
| g_ | 0.0242 | 0.0396 | 0.0005 | 0.0015 | 0.0038 | 0.02 | 0.83 | 0.77 | 0.86 | 0.65 |
| Achromobacter | | | | | | | | | | |
| g_ | 0.0112 | 0.0184 | 0.0002 | 0.0006 | 0.0040 | 0.02 | 0.88 | 0.79 | 0.86 | 0.70 |
| Roseateles | | | | | | | | | | |
| g_ | 0.0383 | 0.0507 | 0.0011 | 0.0022 | 0.0006 | 0.93 | 0.93 | 0.88 | 0.89 | 0.85 |
| Agrobacterium | | | | | | | | | | |
| g_ | 0.0417 | 0.0438 | 0.0071 | 0.0087 | 0.0003 | 0.17 | 0.85 | 0.79 | 0.82 | 0.75 |
| Sphingomonas | | | | | | | | | | |
| g_Kocuria | 0.0006 | 0.0025 | 0.0030 | 0.0035 | 0.0083 | 4.75 | 0.76 | 0.73 | 0.96 | 0.40 |
| g_ | 0.0004 | 0.0011 | 0.0048 | 0.0061 | 0.0048 | 10.88 | 0.81 | 0.77 | 0.89 | 0.60 |
| Desulfovibrio | | | | | | | | | | |
| g_ | 0.0012 | 0.0031 | 0.0172 | 0.0228 | 0.0054 | 14.61 | 0.91 | 0.79 | 0.93 | 0.60 |
| Halomonas | | | | | | | | | | |
| g_ | 0.0003 | 0.0009 | 0.0050 | 0.0051 | 0.0007 | 14.70 | 0.84 | 0.81 | 0.93 | 0.65 |
| Jeotgalicoccus | | | | | | | | | | |

The above description of the present invention is provided only for illustrative purposes, and it will be understood by one of ordinary skill in the art to which the present invention pertains that the invention may be embodied in various modified forms without departing from the spirit or essential characteristics thereof. Thus, the embodiments described herein should be considered in an illustrative sense only and not for the purpose of limitation.

### INDUSTRIAL APPLICABILITY

The method of providing information for diagnosing autism through a bacterial metagenomic analysis according to the present invention may be used for predicting the risk of autism onset and diagnosing autism by performing a bacterial metagenomic analysis using normal individual-derived and subject-derived samples to analyze an increase or decrease in the content of specific bacteria-derived extracellular vesicles.

1. A method of providing information for diagnosing autism, the method comprising:
(a) extracting DNAs from extracellular vesicles isolated from samples from normal individuals and subjects for diagnosis;
(b) performing polymerase chain reaction (PCR) on the extracted DNA using a pair of primers having SEQ ID NO: 1 and SEQ ID NO: 2; and
(c) comparing an increase or decrease in content of bacteria-derived extracellular vesicles of the subject-derived sample with that of the normal individual-derived sample through sequencing of a product of the PCR.

2. The method of claim 1, wherein process (c) comprises comparing an increase or decrease in content of extracellular vesicles derived from one or more bacteria selected from the group consisting of the phylum Armatimonadetes and the phylum Thermi.

3. The method of claim 1, wherein process (c) comprises comparing an increase or decrease in content of extracellular vesicles derived from one or more bacteria selected from the group consisting of the class Fimbriimonadia, the class Aiphaproteobacteria, and the class Deinococci.

4. The method of claim 1, wherein process (c) comprises comparing an increase or decrease in content of extracellular vesicles derived from one or more bacteria selected from the group consisting of the order Fimbriimonadales, the order Rhizobiales, the order Sphingomonadales, and the order Desulfovibrionales.

5. The method of claim 1, wherein process (c) comprises comparing an increase or decrease in content of extracellular vesicles derived from one or more bacteria selected from the group consisting of the family Fimbriimonadaceae, the family Rhizobiaceae, the family Alcaligenaceae, the family Sphingomonadaceae, and the family Desulfovibrionaceae.

6. The method of claim 1, wherein process (c) comprises comparing an increase or decrease in content of extracellular vesicles derived from one or more bacteria selected from the group consisting of the genus Fimbriimonas, the genus Achromobacter, the genus Roseateles, the genus Agrobacterium, the genus Sphingomonas, the genus Kocuria, the genus Desulfovibrio, the genus Halomonas, and the genus Jeotgalicoccus.

7. The method of claim 1, wherein the normal individual and subject sample is urine.

8. The method of claim 1, wherein process (c) comprises comparing an increase or decrease in content of extracellular vesicles derived from one or more bacteria selected from the group consisting of the phylum Armatimonadetes and the phylum Thermi;
extracellular vesicles derived from one or more bacteria selected from the group consisting of the class Fimbriimonadia, the class Aiphaproteobacteria, and the class Deinococci;
extracellular vesicles derived from one or more bacteria selected from the group consisting of the order Fimbriimonadales, the order Rhizobiales, and the order Sphingomonadales, and the order Desulfovibrionales;
extracellular vesicles derived from one or more bacteria selected from the group consisting of the family Fimbriimonadaceae, the family Rhizobiaceae, the family Alcaligenaceae, the family Sphingomonadaceae, and the family Desulfovibrionaceae; or
extracellular vesicles derived from one or more bacteria selected from the group consisting of the genus Fimbriimonas, the genus Achromobacter, the genus Roseateles, the genus Agrobacterium, the genus Sphingomonas, the genus Kocuria, the genus Desulfovibrio, the genus Halomonas, and the genus Jeotgalicoccus.

9. The method of claim 8, wherein in process (c), in comparison with the normal individual-derived sample, an increase in the content of the following is diagnosed as autism:
extracellular vesicles derived from bacteria of the phylum Thermi,
extracellular vesicles derived from bacteria of the class Deinococci,
extracellular vesicles derived from bacteria of the order Desulfovibrionales,
extracellular vesicles derived from bacteria of the family Desulfovibrionaceae, or
extracellular vesicles derived from one or bacteria selected from the group consisting of the genus Kocuria, the genus Desulfovibrio, the genus Halomonas, and the genus Jeotgalicoccus.

10. The method of claim 8, wherein in process (c), in comparison with the normal individual-derived sample, a decrease in the content of the following is diagnosed as autism:
extracellular vesicles derived from bacteria of the phylum Armatimonadetes,
extracellular vesicles derived from one or more bacteria selected from the group consisting of the class Fimbriimonadia and the class Aiphaproteobacteria,
extracellular vesicles derived from one or more bacteria selected from the group consisting of the order Fimbriimonadales, the order Rhizobiales, and the order Sphingomonadales,
extracellular vesicles derived from one or more bacteria selected from the group consisting of the family Fimbriimonadaceae, the family Rhizobiaceae, the family Alcaligenaceae, and the family Sphingomonadaceae, or
extracellular vesicles derived from one or more bacteria selected from the group consisting of the genus Fimbriimonas, the genus Achromobacter, the genus Roseateles, the genus Agrobacterium, and the genus Sphingomonas.

11. A method of diagnosing autism, the method comprising:
(a) extracting DNAs from extracellular vesicles isolated from samples from normal individuals and subjects for diagnosis;
(b) performing polymerase chain reaction (PCR) on the extracted DNA using a pair of primers having SEQ ID NO: 1 and SEQ ID NO: 2; and
(c) comparing an increase or decrease in content of bacteria-derived extracellular vesicles of the subject-derived sample with that of a normal individual-derived sample through sequencing of a product of the PCR.

12. The method of claim 11, wherein process (c) comprises comparing an increase or decrease in content of extracellular vesicles derived from one or more bacteria selected from the group consisting of the phylum Armatimonadetes and the phylum Thermi.

13. The method of claim 11, wherein process (c) comprises comparing an increase or decrease in content of extracellular vesicles derived from one or more bacteria selected from the group consisting of the class Fimbriimonadia, the class Alphaproteobacteria, and the class Deinococci.

14. The method of claim 11, wherein process (c) comprises comparing an increase or decrease in content of extracellular vesicles derived from one or more bacteria selected from the group consisting of the order Fimbriimonadales, the order Rhizobiales, the order Sphingomonadales, and the order Desulfovibrionales.

15. The method of claim 11, wherein process (c) comprises comparing an increase or decrease in content of extracellular vesicles derived from one or more bacteria selected from the group consisting of the family Fimbriimonadaceae, the family Rhizobiaceae, the family Alcaligenaceae, the family Sphingomonadaceae, and the family Desulfovibrionaceae.

16. The method of claim 11, wherein process (c) comprises comparing an increase or decrease in content of extracellular vesicles derived from one or more bacteria selected from the group consisting of the genus Fimbriimonas, the genus Achromobacter, the genus Roseateles, the genus Agrobacterium, the genus Sphingomonas, the genus Kocuria, the genus Desulfovibrio, the genus Halomonas, and the genus Jeotgalicoccus.

17. The method of claim 11, wherein the normal individual and subject sample is urine.

18. The method of claim 11, wherein process (c) comprises comparing an increase or decrease in content of extracellular vesicles derived from one or more bacteria selected from the group consisting of the phylum Armatimonadetes and the phylum Thermi;
extracellular vesicles derived from one or more bacteria selected from the group consisting of the class Fimbriimonadia, the class Alphaproteobacteria, and the class Deinococci;
extracellular vesicles derived from one or more bacteria selected from the group consisting of the order Fimbriimonadales, the order Rhizobiales, and the order Sphingomonadales, and the order Desulfovibrionales;
extracellular vesicles derived from one or more bacteria selected from the group consisting of the family Fimbriimonadaceae, the family Rhizobiaceae, the family Alcaligenaceae, the family Sphingomonadaceae, and the family Desulfovibrionaceae; or
extracellular vesicles derived from one or more bacteria selected from the group consisting of the genus Fimbriimonas, the genus Achromobacter, the genus Roseateles, the genus Agrobacterium, the genus Sphingomonas, the genus Kocuria, the genus Desulfovibrio, the genus Halomonas, and the genus Jeotgalicoccus.

19. The method of claim 18, wherein in process (c), in comparison with the normal individual-derived sample, an increase in the content of the following is diagnosed as autism:
extracellular vesicles derived from bacteria of the phylum Thermi,
extracellular vesicles derived from bacteria of the class Deinococci,
extracellular vesicles derived from bacteria of the order Desulfovibrionales,
extracellular vesicles derived from bacteria of the family Desulfovibrionaceae, or
extracellular vesicles derived from one or bacteria selected from the group consisting of the genus Kocuria, the genus Desulfovibrio, the genus Halomonas, and the genus Jeotgalicoccus.

20. The method of claim 18, wherein in process (c), in comparison with the normal individual-derived sample, a decrease in the content of the following is diagnosed as autism:
extracellular vesicles derived from bacteria of the phylum Armatimonadetes,
extracellular vesicles derived from one or more bacteria selected from the group consisting of the class Fimbriimonadia and the class Aiphaproteobacteria,
extracellular vesicles derived from one or more bacteria selected from the group consisting of the order Fimbriimonadales, the order Rhizobiales, and the order Sphingomonadales,
extracellular vesicles derived from one or more bacteria selected from the group consisting of the family Fimbriimonadaceae, the family Rhizobiaceae, the family Alcaligenaceae, and the family Sphingomonadaceae, or
extracellular vesicles derived from one or more bacteria selected from the group consisting of the genus Fimbriimonas, the genus Achromobacter, the genus Roseateles, the genus Agrobacterium, and the genus Sphingomonas.
